(19) United States
(12) Patent Application Publication
(10) Pub. No.: US 2012/0059236 A1
(43) Pub. Date: Mar. 8, 2012

(54) IMPLANTABLE DEVICE FOR REAL-TIME MONITORING OF GLYCEMIA AND DOSAGE

(76) Inventor: Juliana Castro Dourado Pinezi, Gioania (BR)

(21) Appl. No.: 12/877,384

(22) Filed: Sep. 8, 2010

(51) Int. Cl. A61B 5/145

(52) U.S. Cl. 600/365

(57) ABSTRACT

An implantable device for constant collection and dosage of glycemia with real-time monitoring, with no need of external collection by punctioning which is constituted by a metal compartment, hermetically sealed with the cap, containing at least a micro-computerized mini-center fed by the battery or similar, which is externally rechargeable. The device contains a sensor which collects a portion of the circulating blood and reads glycemia levels in real time, sending reading information through the transmission component. The device is connected to an inlet catheter with its collecting edge duly implanted by means of punctioning at the subclavian vein, to deviate a small flow of blood to pass through within said dosage and monitoring device, thus allowing the sensor to verify the glycemic dosage and following its natural course by exiting the exit catheter through the outlet edge, thus returning to the blood current within said subclavian vein.

### FIELD OF THE INVENTION

The present invention refers to an implantable device for constant collection and dosage of glycemia with real-time monitoring and with no need of external collections by vein puncture.

### BACKGROUND OF THE INVENTION

Scientific studies prove that the level of glucose is determined by using vein plasma or serum, as measured by the glucose oxidase method as used in laboratories. For clinical purposes, both the level in plasma and in serum are similar enough to be interchangeable. However, currently available glucose meters measuring capillary glycemia only guarantee 15% exact results over the values as measured in plasma or serum by clinical laboratories.

The collection of peripheral blood for laboratory tests of glycemia dosage is made at the surface veins of upper members, usually at the basilic vein or the cephalic vein. The dosage on that blood is considered as the golden standard to which glucose meter readings are compared for a periodical quality control, usually each six months. Brachial veins are joined to the basilic vein to form the auxiliary vein, which the other surface vein of the upper member (cephalic vein) flows to. The auxiliary vein becomes the subclavian vein after crossing the first rib. For having a larger diameter, for being more easily accessed in surgeries and for being the first choice when a central vein access is indicated (since this is the site with less risk of thrombosis and infections related to catheters in comparison with internal femoral and jugular veins), the subclavian vein is the ideal candidate for the implantation of implantable catheters of the device at issue. Therefore, the reading of the device as presented herein will be made by using the same blood collected in a laboratory and considered as a golden standard for the dosage of glycemia.

The use of new technologies in the monitoring of glycemia has been a challenge for specialists and corporations. Many of them do not hold a license in some countries, while others have been taken from the market when they started to be used, due to problems that were verified. An example was the quick commercialization of the Pendra device, which was subsequently taken off from the European market. Wentholt et al disclosed in an article (Diabetologia 2005; 48: 1055-1058) what would be the development of a non-invasive glycemia sensor, which turned out to be a danger for diabetic patients since its average reading error was 52%.

Another non-invasive device having problems with humidity which was taken off from the market was Cygnus Glucowatch.

Currently, one equipment meeting clinical interests is Medtronic Diabetes MiniMed CGMS—continuous glucose monitoring. That device uses a needle to measure glucose in the interface by means of the reaction between glucose-oxidase and the immobilized enzyme in the electrode as subcutaneously inserted, enabling the observation of fluctuating rates of glycemia throughout the day. The sensor lasts about three days and receives the results, downloading them to make registration comparisons. In some cases, they are used for seven days. The values are joined and sum a total each five minutes. The device has a small electrode connected to a router downloading data to another equipment. When installed, it needs one hour of pre-heating to then obtain a glycemia value to be included. There are about 280 measurements per day. The patient makes a log with feeding and carbohydrate counts to enable the doctor to evaluate the reason of oscillations in glycemia. The use of CGMS serves to improve glycemia control, detect and reduce the risk of hypoglycemic events and improve intensive insulin delivery schemes. However, considering the discomfort produced and how it is implanted, it cannot be used for long periods to substitute traditional auto monitoring with the glucose meter examining the blood drop as obtained by the lancet at the edge of the fingers (capillary glycemia). In the same way, Medtronic MiniMed long-term IV sensor is still under development, having an intravessel electrode linked to an insulin pump.

Equally, another available subcutaneous glucose sensor is GlucoDay sensor (Menarini Diagnostics, Firenze, Italy). Said sensor is based on microdyalisis, wherein the catheter is connected to a walkman-sized device containing a glucose biosensor. Glucose is reported each three minutes.

On the other hand, HypoMon is just a system for hypoglycemia detection and alarm (glycemia<45 mg/dl). The device uses four electrodes in contact with the skin and a handheld computer with wireless communication system (no wires). It continuously monitors heart frequency and QT intervals and detects physiological signs of hypoglycemia by means of an algorithm.

In the same fashion, a device known as Freestyle Navigator is also known, using a subcutaneous needle and a transmitting device fixed to the skin by a sticker held for various days. By means of wireless communication, it sends the dosage of glycemia minute by minute to a pager-shaped receiver. There is also the DexCom implantable system, wherein the sensor is installed subcutaneously and the data is sent to an external receiver. However, these options are still in clinical evaluation or development and are inconveniently painful and/or expose the diabetic patient to the society as an ill individual with various accessories fixed to the body, limiting its privacy.

Furthermore, even among so many options, no device has yet proved to be sufficiently accurate to substitute auto monitoring with glucose meters. Diabetic patients remain depending on said auto monitoring measuring capillary glycemia by using lancets damaging finger edges to obtain blood drops used for analysis. These patients consider the method as painful, constraining, expensive, anti-hygienic and inconvenient, as well as the traditional methods causing large discomfort to patients.

### BRIEF DESCRIPTION OF THE DRAWINGS

A complete understanding of the present invention may be obtained by reference to the accompanying drawings, when considered in conjunction with the subsequent detailed description, in which:

FIG. 1 shows a front view of the present invention duly implanted to the subclavian vein;

FIG. 2 shows an internal view of the present invention of FIG. 1; and

FIG. 3 shows the subclavian vein (VS) itself, wherein the device of the present invention should be implanted, as well as the axillary (VA), cephalic (VC), basilic (Vb) and brachial veins (Vbq).

### DESCRIPTION OF THE PREFERRED EMBODIMENT

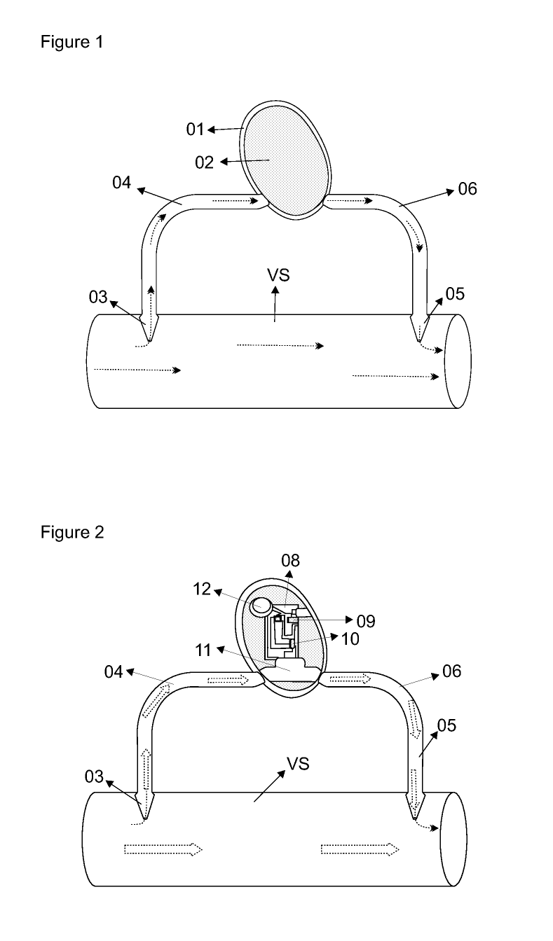

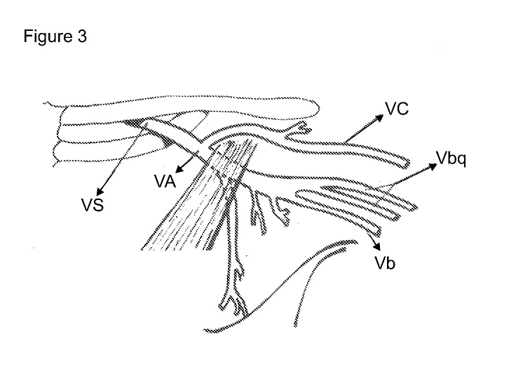

Referring to FIGS. 1 and 2, the present invention comprises a metal compartment 01, hermetically sealed with the cap 02, containing at least one micro computerized minicentral 08 fed by the battery 12 or similar, externally rechargeable, and will also contain a sensor 11 which is responsible to effect glycemia readings in real time, by sending reading information by means of the transmission component 10.

With that purpose, said implantable device will be connected to an inlet catheter 04 with its collecting edge 03, which will be implanted by means of punctioning at the subclavian vein (VS), so to deviate a small flow of blood which will pass through within said dosage and monitoring device, thus allowing the sensor 11 to verify the glycemia dosage and follow its natural course, exiting through the outlet catheter 06 at the outlet edge 05, thus returning to the blood flow inside said subclavian vein (VS).

Therefore, the external components which will remain in contact with the internal organs of the patient will be manufactured and covered with titanium or another similar metal to vessel prosthesis allowing for long use for various years and presenting less risk of rejection.

We should strongly highlight that the result of glycemia analysis as effected by said device will be sent by means of radio frequency waves (or similar) to an external receiver, which may have various shapes and sizes, preferably that of an object for personal use, such as a wristwatch, wherein the patient will be able to receive and monitor glycemia data as continuously measured, minute by minute, besides containing the schedule to advise the patient, by means of an alarm signal, in case of any abnormal situation, be it in case of hypoglycemia or even hyperglycemia, since the micro computerized center 08 will be scheduled with minimum and maximum dosage scales for glycemia, so to send a warning to the receiver and, therefore, the patient may take his or her decisions.

Since other combinations, modifications and changes varied to fit particular operating requirements and environments will be apparent to those skilled in the art, the invention is not considered limited to the chosen preferred embodiments for purposes of this disclosure, but covers all changes and modifications which do not constitute departures from the true spirit and scope of this invention.

Having thus described the invention, what is desired to be protected by Letters Patent is presented in the subsequently appended claims.

What is claimed is:

1. An implantable device for real-time monitoring and dosage of glycemia comprising a hermetically sealed compartment with a cap having at least a micro computerized minicenter supplied by the battery or similar which is externally rechargeable, and a sensor for reading and analysis and a transmitting component to send the data through wireless waves.

2. The implantable device in accordance with claim 1, characterized by reading glycemia in real time, with no discharge of blood, directly collected by an inlet catheter with a collecting edge, duly implanted by means of puncture in the subclavian vein (VS) so to deviate a small flow of blood to pass through within said monitoring and dosage device, thus allowing the sensor to verify glycemia dosage and allowing the blood to follow its natural course exiting through the outlet catheter through the outlet edge, thus returning to the blood flow inside said Subclavian Vein (VS).

3. The implantable device in accordance with claim 1, characterized by containing a data reception device allowing the patient to both monitor, minute by minute, the glycemia rate and be advised by means of a sound or visual alarm in case of any change indicating hypoglycemia or even hyperglycemia.

4. The implantable device in accordance with claim 2, characterized by containing a data reception device allowing the patient to both monitor, minute by minute, the glycemia rate and be advised by means of a sound or visual alarm in case of any change indicating hypoglycemia or even hyperglycemia.
